(19) United States
(12) Patent Application Publication
(10) Pub. No.: US 2014/0271926 A1
(43) Pub. Date: Sep. 18, 2014

(54) METHODS OF USE OF GLUTAMINE SYNTHETASE INHIBITORS

(71) Applicant: CALIFORNIA INSTITUTE OF TECHNOLOGY, Pasadena, CA (US)

(72) Inventors: Thang V. Nguyen, Pasadena, CA (US); Raymond J. Deshaies, Pasadena, CA (US)

(21) Appl. No.: 14/207,510

(22) Filed: Mar. 12, 2014

Related U.S. Application Data
(60) Provisional application No. 61/778,029, filed on Mar. 12, 2013.

(51) Int. Cl. A61K 31/713; C12Q 1/25; C12N 15/113; A61K 31/662; A61K 33/18; A61K 38/08; A61K 38/06; A61K 31/198; G01N 33/573; A61K 31/454

(52) U.S. Cl. CPC A61K 31/713; G01N 33/573; C12Q 1/25; C12N 15/1137; A61K 31/454; A61K 33/18; A61K 38/08; A61K 38/06; A61K 31/198; A61K 31/662
USPC 424/667; 435/4; 435/7.4; 514/44 A; 514/323; 514/19.2; 514/562; 514/119

(57) ABSTRACT

A method of treating neoplastic growth in a subject includes administering a glutamine synthetase (GS) inhibitor to the subject having neoplastic growth. A glutamine synthetase inhibitor may be administered in combination with thalidomide, lenalidomide and/or pomalidomide. Responsiveness to thalidomide, lenalidomide or pomalidomide therapy is determined by the expression levels of glutamine synthetase in neoplastic cells.

### CROSS-REFERENCE TO RELATED APPLICATION(S)

The present application claims priority to and the benefit of U.S. Provisional Application Ser. No. 61/778,029 filed on Mar. 12, 2013, the entire contents of which are incorporated herein by reference.

### STATEMENT REGARDING FEDERALLY SPONSORED RESEARCH OR DEVELOPMENT

This invention was made with government support under DA032474 awarded by the National Institutes of Health. The government has certain rights in the invention.

### FIELD

This disclosure is directed to the use of glutamine synthetase inhibitors for treating neoplastic growth, such as cancer.

### BACKGROUND

Thalidomide and its closely related derivatives, lenalidomide and pomalidomide, have been used effectively to treat patients afflicted with leprosy, mylodysplastic syndrome, and multiple myeloma. (Teo et al., 2002, Microbes and infection/Institut Pasteur, 4:1193; Komrokji et al., 2012, Curr Pharm Des, 18:3198; and Zhu et al., 2013, Leukemia & Lymphoma, 54:683, the entire contents of all of which are herein incorporated by reference.) The efficacy of these drugs is thought to be due to their ability to suppress the production of the immunomodulatory agent tumor necrosis factor-alpha (TNF-α), and therefore these compounds have been collectively referred to as immunomodulators (IMiDs). (Sampaio et al., 1991, J. Exp Med, 173: 699, the entire contents of which are herein incorporated by reference.) The teratogenic effect of thalidomide has been linked to its binding to the protein cereblon (CRBN) which is a putative substrate receptor subunit for a cullin RING ubiquitin ligase 4 (CRL4) complex. (Ito et al., 2010, Science, 327:1345 and Angers et al., 2006, Nature, 443:590, the entire contents of both of which are herein incorporated by reference.) Subsequent work has linked CRBN to the anti-tumor necrosis factor (TNF)-α and anti-myeloma effects of IMiDs. (Lopex-Girona et al., 2012, Leukemia, 26:2326 and Zhu et al., 2011, Blood, 118:4771, the entire contents of both of which are herein incorporated by reference.) However, the direct substrates of CRBN and how IMiDs influence their degradation remain unknown.

### SUMMARY

In some embodiments of the present invention, a method of treating neoplastic growth in a subject includes administering a composition including a glutamine synthetase (GS) inhibitor to the subject having neoplastic growth. In some embodiments the neoplastic growth is a cancer.

In some embodiments of the present invention, the method of treating neoplastic growth in a subject includes administering a glutamine synthetase (GS) inhibitor and administering thalidomide, lenalidomide and/or pomalidomide.

In some embodiments of the present invention, the glutamine synthetase (GS) inhibitor is selected from GS anti-sense mRNA, GS siRNA, GS shRNA, GS miRNA, or GS oligonucleotides.

In some embodiments of the present invention, a method of identifying a response to immunomodulatory (IMiD) and glutamine synthetase (GS) inhibitor therapy in a subject includes measuring the level of glutamine synthetase (GS) protein in the subject before administering the IMiD therapy in combination with administering a GS inhibitor to the subject, and measuring the level of GS protein in the subject after administering the IMiD therapy and GS inhibitor to the subject, where a reduction in the level of GS protein in the subject after IMiD therapy is indicative of a response to the IMiD therapy.

In some embodiments of the present invention, a method of identifying the capability of a subject having neoplastic cell growth to respond to immunomodulatory drug therapy, includes determining the amount of glutamine synthetase expression in the neoplastic cell growth of the subject, and determining the amount of glutamine synthetase expression in normal cells of the subject; where an increase in the expression of glutamine synthetase in the neoplastic cell growth compared to the expression of glutamine synthetase in normal cells of the subject indicates that the neoplastic cell growth of the subject is capable of responding to immunomodulatory drug therapy, and wherein if the subject is capable of responding to immunomodulatory drug therapy, the method further comprising administering a glutamine synthetase inhibitor to the subject.

In some embodiments of the present invention, a composition for inhibiting neoplastic cell growth includes thalidomide, lenalidomide and/or pomalidomide in combination with a glutamine synthetase inhibitor.

### BRIEF DESCRIPTION OF THE DRAWINGS

FIG. 1 is a graph showing the increase (measured as fold increase) of interaction of the indicated protein in an immunoprecipitation (IP) of ^{Flag}CRBN expressed in HEK293 T cells using anti-Flag resin, according to embodiments of the present invention.

FIG. 2 is an immunoblot of an SDS-PAGE gel of cell lysate and selected ^{Flag}CRBN immunoprecipitates from FIG. 1 in the presence and absence of thalidomide, according to embodiments of the present invention.

FIG. 3 is an immunoblot of the indicated proteins on an SDS-PAGE gel loaded with the total cell lysate and the bound fractions in an IP of endogenous CRBN in MM.1S cells in the presence and absence of lenalidomide, according to embodiments of the present invention.

FIG. 4 is an immunoblot of the indicated proteins on an SDS-PAGE gel loaded with the cell lysate and bound fractions of a TUBE2 resin in HEK293T cells in the presence and absence of each of lenalidomide and MG132, according to embodiments of the present invention.

FIG. 5 is an immunoblot of the indicated endogenous proteins on an SDS-PAGE gel loaded with total cell lysates of MM.1S cells grown in the presence of 2 mM glutamine and the indicated doses of pomalidomide or lenalidomide for 24 hours, according to embodiments of the present invention.

FIG. 6 is a graph showing the relative abundance of GLUL mRNA in MM.1S cells treated with 30 μM lenalidomide for the indicated time (hours), as determined by RT-PCR and normalized to GAPDH mRNA, according to embodiments of the present invention.

FIG. 7 is an immunoblot of GS and GAPDH on an SDS-PAGE gel loaded with total cell lysates of MM.1S cells grown in the presence of the indicated doses of pomalidomide and 5-NH₂, according to embodiments of the present invention.

FIG. 8 is an immunoblot of the indicated proteins on an SDS-PAGE gel loaded with total cell lysates of MM.1S cells grown in the presence of 2 mM glutamine and 100 μM lenalidomide for 24 hours, according to embodiments of the present invention.

FIG. 9 is an immunoblot of the indicated proteins on an SDS-PAGE gel loaded with total cell lysates of MM.1S cells pre-treated with 100 μM pomalidomide for 1 hour, followed by addition of 100 μg/ml cycloheximide (CHX), according to embodiments of the present invention.

FIG. 10 is an immunoblot of the indicated proteins on an SDS-PAGE gel loaded with total cell lysates of MM.1S cells grown in the presence of 2 mM glutamine, pre-treated with 100 μm lenalidomide for 1 hour where indicated, followed by addition of 100 μg/ml cycloheximide (CHX) for the indicated time in hours (hr), according to embodiments of the present invention.

FIG. 11 is an immunoblot of the indicated proteins on an SDS-PAGE gel loaded with total cell lysates of MM.1S cells grown in the presence or absence of 0.5 μM MG132 as indicated, and the presence or absence of 100 μM lenalidomide, as indicated, according to embodiments of the present invention.

FIG. 12 is an immunoblot of the indicated proteins on an SDS-PAGE gel loaded with total cell lysates from MM.1S cells expressing a control shRNA knockdown construct or CRBN lentiviral shRNA knockdown construct and grown for 24 hours in the presence of the indicated does of lenalidomide, according to embodiments of the present invention.

FIG. 13A is an immunoblot of HA or Flag proteins as indicated on an SDS-PAGE gel loaded with total cell lysates (input) or the immunoprecipitated fractions from an IP of ^{Flag}GS in HEK293T cells transfected with a plasmid expressing ^{Flag}GS and infected with either control lentivirus or lentiviruses expressing CRBN shRNAs; the cells were then grown in the presence or absence of ^{HA}Ubiquitin (^{HA}Ub) and/or MG 132 as indicated, according to embodiments of the present invention.

FIG. 13B is an immunoblot of the indicated proteins (CRBN and GAPDH) on an SDS-PAGE gel loaded with the total cell lysates shown in FIG. 13A to control for the infections of the non-target shRNA and the CRBN shRNA, according to embodiments of the present invention.

FIG. 14 is an immunoblot of the indicated proteins (GST and Flag) on an SDS-PAGE gel loaded with samples after GST or GST-GS was incubated with CRBN in the presence or absence of thalidomide as indicated, followed by precipitation by GST pulldown, according to embodiments of the present invention.

FIG. 15 is an immunoblot of the indicated proteins on an SDS-PAGE gel. HEK293T that express ^{Flag}CRBN were transiently transfected with plasmids that express ^{HA}DDB1, ^{V5}CUL4A, and ^{HA}RBX1 (lanes 2, 3, and 4) or transfected with empty vector (lane 1); after immunoprecipitation with Flag resin, in vitro ubiquitylation of endogenous, co-precipitated GS was carried out for 2 hours at 30° C. in the presence or absence of E1, E2s, and ubiquitin (Ub), and where indicated methylated ubiquitin (Me-Ub); total cell lysates and the final Flag precipitates were separated by SDS-PAGE gel, according to embodiments of the present invention.

FIG. 16 is an immunoblot of the indicated proteins on an SDS-PAGE gel loaded with cell lysates of MM.1S cells after transduction for 48 hours with control (CT) shRNA lentivirus or CRBN shRNA lentivirus as indicated, according to embodiments of the present invention.

FIG. 17A is an immunoblot of the indicated proteins on an SDS-PAGE gel loaded with cell extracts from HEK293T cells transduced with six different GS shRNA-expressing lentiviruses (1-6) and a non-target shRNA-expressing lentivirus (CT), according to embodiments of the present invention.

FIG. 17B is an immunoblot of the indicated proteins on an SDS-PAGE gel loaded with cell extracts from MM.1S cells transduced with four different GS shRNA-expressing lentiviruses (GS_{—}2, GS_{—}5, GS_{—}3, GS_{—}4) and a non-target shRNA-expressing lentivirus (CT), according to embodiments of the present invention.

FIG. 18 is a graph showing a cell count at Day 0, Day 3, and Day 6 of MM.1S cells transduced with lentiviral vectors that express either control (CT) shRNA or GS shRNA (GS_{—}3) with puromycin selection for 6 days in complete medium containing 2 mM glutamine; cell number was quantified by staining with 0.4% Trypan blue at the indicated times, according to embodiments of the present invention.

FIG. 19 is a photograph showing the abnormal morphology of GS-depleted MM.1S cells and control MM.1S cells of FIG. 18, except that cells were shifted to medium containing 0.5 mM glutamine for 48 hours prior to being photographed at 10×, according to embodiments of the present invention.

FIG. 20 is a graph showing the percentage of dead (i.e., Trypan blue) MM.1S cells after growth in a medium containing 0.5 mM glutamine for 48 or 96 hours as indicated, and staining with 0.4% Trypan blue, according to embodiments of the present invention.

FIG. 21 is an immunoblot of the indicated proteins on an SDS-PAGE gel loaded with total cell lysates of the MM.1S cells of FIG. 18, transduced with lentiviral vectors that express either control (CT) shRNA or GS shRNA3 (shGS_{—}3) were subjected to puromycin selection for 1 week in complete medium containing 2 mM glutamine, followed by culturing in 0.5 mM glutamine for 48 hours; protein extracts were analyzed by immunoblotting with caspase 3, PARP (apoptosis biomarkers), GS or GAPDH (control) antibodies, according to embodiments of the present invention.

FIG. 22 is a graph showing percent cell viability (normalized to DMSO) of MM.1S cells transduced with shCT (control) lentivirus, shGS_{—}3 lentivirus, or shGS_{—}4 lentivirus incubated with DMSO, 0.5 μm lenalidomide, or 5 μM lenalidomide as indicated, according to embodiments of the present invention.

FIG. 23 is a graph showing percent cell viability (normalized to DMSO) of MM.1S cells transduced with control shCT lentivirus or shGS_{—}5 lentivirus incubated with increasing amounts of lenalidomide as indicated, according to embodiments of the present invention.

FIG. 24 is a graph showing percent cell viability (normalized to DMSO) of MM.1S cells transduced with shCT (control) lentivirus, shGS_{—}3 lentivirus, or shGS_{—}4 lentivirus and cultured for 1 week with puromycin selection in a medium containing 2 mM glutamine, followed by a shift to a medium containing 0.5 mM glutamine for 72 hours in the presence of DMSO, 50 nM pomalidomide, or 500 nM pomalidomide, according to embodiments of the present invention.

FIG. 25 is a graph showing percent cell viability (normalized to DMSO) of MM.1S cells transduced with shCT (control) lentivirus, shGS_{—}3 lentivirus, or shGS_{—}4 lentivirus and cultured for 1 week with puromycin selection in a medium containing 2 mM glutamine, followed by a shift to a medium containing 0.5 mM glutamine for 72 hours in the presence of increasing amounts of pomalidomide as indicated, according to embodiments of the present invention.

FIG. 26 is an immunoblot of the indicated proteins on an SDS-PAGE gel loaded with total cell lysates of MM.1S cells transduced with shCT (control) lentivirus or shGS_{—}3 lentivirus and grown in a medium containing 2 mM glutamine with puromycin selection for 1 week, followed by a shift for 72 hours in a medium containing 0.5 mM glutamine and increasing amounts of pomalidomide as indicated, according to embodiments of the present invention.

### DETAILED DESCRIPTION

Aspects of the present invention are directed to inhibiting glutamine synthetase (GS) in neoplastic cells. Inhibition of GS may inhibit the proliferation of neoplastic cells. As shown herein, GS is an endogenous substrate of CRL4^{CRBN}, and in the presence of immunomodulators (IMiDs) (thalidomide, lenalidomide, and pomalidomide), GS binding to CRBN may be enhanced, leading to an increase in GS ubiquitylation and degradation. Moreover, knockdown (i.e., inhibition) of GS may block proliferation of neoplastic growth as shown herein in myeloma cells. Inhibition of GS may also enhance sensitivity to IMiD treatment. In this way, inhibition of GS may confer an inhibition of neoplastic cell growth when administered alone to neoplastic cells.

Additionally, because of the potential for increased sensitivity to IMiD therapy, the administered effective dosage of thalidomide, lenalidomide, and pomalidomide may be significantly decreased when an IMiD is used in combination with an inhibitor of GS. Administration of lower dosages allows for potentially fewer side effects. In some embodiments of the present invention, a composition for inhibiting neoplastic growth includes thalidomide, lenalidomide, and/or pomalidomide in combination with a GS inhibitor. As shown herein, the amount of IMiD required for growth inhibition is reduced when added in combination with a GS inhibitor.

As used herein, the terms “inhibition,” “inhibiting,” and “inhibit” and like terms, refer to preventing growth and/or decreasing the rate of growth. In the context of cell growth, these terms refer to preventing or decreasing cell growth. In the context of glutamine synthetase, inhibition, inhibiting, and inhibit refer to the substantial elimination or decrease in expression of or activity of glutamine synthetase. As used herein, the terms “substantially” and “substantial” are used as terms of approximation and not as terms of degree.

As used herein “neoplastic” refers to unregulated growth. An example of neoplastic growth includes growth of cancer cells. For example, neoplastic growth may refer to unregulated growth, such as cancer. Neoplastic transformation is accompanied by increases in nucleotide and protein synthesis, and the high rates of protein synthesis in rapidly growing cells require a continuous supply of both essential and non-essential amino acids. Glutamine is currently understood to be required for all known types of cancer growth. (Medina, 2001, Jn Nutrition, 131:2539S, the entire contents of which are herein incorporated by reference.) Non-limiting examples of cancer types include myeloma, bladder cancer, breast cancer, colon cancer, rectal cancer, endometrial cancer, renal cell cancer, leukemia, lung cancer, melanoma, non-Hodgkin's lymphoma, Hodgkin's lymphoma, pancreatic cancer, prostate cancer, and thyroid cancer.

Thalidomide and its closely related derivatives, lenalidomide and pomalidomide are referred to as immunomodulators (IMiDs), which are shown herein to have a synergistic effect in combination with a glutamine synthetase (GS) inhibitor on inhibiting the proliferation of cells expressing GS, such as multiple myeloma cells. In some embodiments of the present invention, a method of identifying a response to immunomodulatory drug (IMiD) therapy in a subject includes measuring the level of glutamine synthetase (GS) protein in the subject before administering the IMiD and GS inhibitor therapy to the subject, followed by measuring the level of GS protein in the subject after administering the IMiD and GS inhibitor therapy to the subject. With these calculations, an observed reduction in the level of GS protein in the subject after IMiD and GS inhibitor therapy is indicative of a response to the IMiD and GS inhibitor therapy.

Additionally, in some embodiments of the present invention, a method of identifying the capability of a subject having neoplastic cell growth to respond to immunomodulatory (IMiD) and GS inhibitor therapy includes determining the amount of glutamine synthetase expression in the neoplastic cell growth of the subject, followed by determining the amount of glutamine synthetase expression in normal cells of the subject. With these calculations, an increase in the expression of glutamine synthetase in the neoplastic cells compared to the expression of glutamine synthetase in the normal cells of the subject indicates that the neoplastic cell growth of the subject is capable of responding to IMiD and GS inhibitor therapy. Furthermore, a subject having a neoplastic growth that is capable of responding to IMiD and GS inhibitor therapy includes administering the IMiD and GS inhibitor to the subject.

In some embodiments of the present invention, an inhibitor of glutamine synthetase may be administered to neoplastic cells either in vitro or in vivo. Examples of glutamine synthetase inhibitors are known in the art. Non-limiting examples of GS inhibitors include GS interfering RNA (RNAi) GS anti-sense mRNA, GS small interfering (si) RNA, GS short hairpin (sh)RNA, GS micro(mi)RNA, and GS oligonucleotides (DNA or RNA).

Methods for synthesizing siRNA are known in the art and for example, are disclosed in Kumar et al., Nature 448: 39-43, 2007; Pulford et al., PLoS One 5:e11085, 2010; and Rohn et al., J. Drug Target, 20: 381-388, 2012, the entire contents of all of which are incorporated herein by reference. Methods for synthesizing shRNA or microRNA are known in the art and for example, are disclosed in Hwang do et al., Biomaterials, 32: 4968-4975, 2011, the entire contents of which are incorporated herein by reference. Methods for synthesizing oligonucleotides (DNA or RNA) are known in the art and for example, are disclosed in Pardridge, Jpn J Pharmacol, 87:97-103, 2001, the entire contents of which are incorporated herein by reference. Methods for synthesizing modified oligonucleotides (e.g., DNA or RNA) are known in the art and for example, are disclosed in Pardridge, 2001, supra;

Non-limiting examples of a glutamine synthetase inhibitor include methionine sulfoximine, methionine sulfone, phosphinothricin, tabtoxinin-b-lactam, methionine sulfoximine phosphate, alpha-methyl methionine sulfoximine, alpha-ethyl methionine sulfoximine, ethionine suloximine, alpha-methyl ethionine sulfoximine, prothionine sulfoximine, alpha-methyl prothionine sulfoximine, gamma-hydroxy phosphinothricin, gamma-methyl phosphinothricin, gamma-acetoxy phosphinothricin, alpha-methyl phosphinothricin, alpha-ethyl phosphinothricin, cyclohexane phosphinothricin, cyclopentane phosphinothricin, tetrhydrofuran phosphinothricin, s-phosphonomethylhomocysteine, s-phosphonomethyl homocysteine sulfoxide, s-phosphonomethyl homocysteine sulfone, 4-(phosphonoacetyl)-L-alpha-aminobutyrate, threo-4-hydroxy-D-glutamic acid, threo-4-fluoro-D,L-glutamic acid, erythro-4-fluoro-D,L-glutamic acid, 2-amino-4-[(phosphonomethyl)hydroxyphosphinyl)]butanoic acid, alanosine, 2-amino-4-phosphono butanoic acid, 2-amino-2-methyl-4-phosphono butanoic acid, 4-amino-4-phosphono butanoic acid, 4-amino-4-(hydroxymethylphosphinyl)butanoic acid, 4-amino-4-methyl-4-phosphono butanoic acid, 4-amino-4-(hydroxymethylphosphinyl)-4-methyl butanoic acid, 4-amino-4 phosphono butanamide, 2-amido-4-phosphono butanoic acid, 2-methoxycarbonyl-4-phosphono butanoic acid, methyl 4-amino-4-phosphono butanoate, oxetin, IF7 peptide, or IF17 peptide, as disclosed in Eisenberg et al., 2000, Biochim. Biophys. Acta, 1477:122-145, the entire contents of which are herein incorporated by reference.

In some embodiments of the present invention, a composition including a GS inhibitor or a composition including thalidomide, lenalidomide, and/or pomalidomide in combination with a GS inhibitor as disclosed herein, can be prepared to be delivered in a “prodrug” form. The term “prodrug,” as used herein, indicates a therapeutic agent that is prepared in an inactive form that is converted to an active form (i.e., drug) within the body or cells thereof by the action of endogenous enzymes or other chemicals and/or conditions.

As used herein, the terms “composition” or “pharmaceutical composition” are used interchangeably and refer to compositions or formulations that in addition to the active ingredient (e.g., GS inhibitor), may also include an excipient, such as a pharmaceutically acceptable carrier, that is conventional in the art and that is suitable for administration to living organisms, including mammals (e.g., humans), and cells thereof Such compositions may be specifically formulated for administration via one or more of a number of routes, including but not limited to, oral, parenteral, intravenous, intraarterial, subcutaneous, intranasal, sublingual, intraspinal, intracerebroventricular, or the like. Cells may be administered with the GS inhibitor composition as disclosed herein for example, for therapeutic or diagnostic purposes. These cells may be part of a subject, e.g., a living organism. The cells may also be cultured, for example, cells that are a part of an assay for screening potential pharmaceutical compositions or the efficacy of a therapy; and the cells may be a part of a transgenic animal for research purposes. In addition, compositions for topical (e.g., oral mucosa, respiratory mucosa) and/or oral administration may form solutions, suspensions, tablets, pills, capsules, sustained-release formulations, oral rinses, or powders, as known in the art. The compositions also can include stabilizers and/or preservatives. For examples of carriers, stabilizers and adjuvants, see Lippincott Williams & Wilkins, (2006) Remington: The Science and Practice of Pharmacy, 21st Ed, Editor David B. Troy, the entire contents of which are herein incorporated by reference.

GS inhibitor compositions as disclosed herein may be administered by any convenient route, including parenteral, enteral, mucosal, topical, e.g., subcutaneous, intravenous, topical, intramuscular, intraperitoneal, transdermal, rectal, vaginal, intranasal or intraocular. In one embodiment, the delivery is by oral administration of the composition formulation. In one embodiment, the delivery is by intranasal administration of the composition. Along these lines, intraocular administration is also possible. In another embodiment, the delivery means is by intravenous (i.v.) administration of the composition, which is especially advantageous when a longer-lasting i.v. formulation is desired. Suitable formulations can be found in Remington's Pharmaceutical Sciences, 16th and 18th Eds., Mack Publishing, Easton, Pa. (1980 and 1990), and Introduction to Pharmaceutical Dosage Forms, 4th Edition, Lea & Febiger, Philadelphia (1985), each of which is incorporated herein by reference.

The GS inhibitor compositions, as disclosed herein, may be administered in therapeutically effective amounts. The GS inhibitor composition herein may be administered along with a pharmaceutically acceptable material—such as an excipient, carrier, stabilizer, and/or adjuvant. A therapeutically effective amount means the amount necessary, at least partly, to attain the desired effect of inhibiting neoplastic growth. Such amounts will depend on the particular condition being treated, the severity of the condition and individual patient parameters including age, physical condition, size, weight and concurrent treatment. These factors are well known to those of ordinary skill in the art and can be addressed with no more than routine experimentation. If possible a maximum dose should be administered. The maximum dose is the highest safe dose according to sound medical judgment. It will be understood by those of ordinary skill in the art, however, that a lower dose or tolerable dose may be administered for medical reasons, psychological reasons or for any other reason.

As used herein, the term “pharmaceutically acceptable carrier” refers to any pharmaceutically acceptable means to mix and/or deliver the GS inhibitor composition to a living organism. Examples of pharmaceutically acceptable carriers include liquids, solid fillers, diluents, excipients, solvents and/or encapsulating materials, involved in sustaining, carrying and/or transporting the subject agents (e.g., the GS inhibitor) from one organ, or portion of the body, to another organ, or portion of the body. The carrier material must be “acceptable” in the sense of being compatible with the other ingredients of the formulation and is compatible with administration to the particular living organism, for example a human or the cells of a human. For the clinical use of the methods of the present invention, the GS inhibitor composition of the invention is formulated into pharmaceutical compositions or pharmaceutical formulations for parenteral administration, e.g., intravenous; mucosal, e.g., intranasal; enteral, e.g., oral; topical, e.g., transdermal; ocular, e.g., via corneal scarification or other mode of administration. The pharmaceutical composition contains a compound of the invention in combination with one or more pharmaceutically acceptable materials, for example, a carrier. The carrier may be in the form of a solid, semi-solid or liquid diluent, cream or a capsule. The amount of GS inhibitor in the pharmaceutical composition according to embodiments of the present invention may be between 0.1-95% by weight of the preparation, for example, between 0.2-20% by weight in preparations for parenteral use, and between 1 and 50% by weight in preparations for oral administration.

As used herein, the term “parenteral administration” and “administered parenterally” means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically,” “peripheral administration” and “administered peripherally” as used herein mean the administration of a compound, drug or other material other than directly at a site of infection, such that it enters a system of the living organism (e.g., the circulatory system, the respiratory system, or through the skin) and, thus, is subject to metabolism and other like processes.

As used herein, the terms “administering” and “introducing” are used interchangeably and refer to the placement of the pharmaceutical composition including an GS inhibitor composition according to some embodiments of the present invention, into a living organism or cells thereof by a method or route which results in at least partial localization of the GS inhibitor at a desired site. The GS inhibitor composition according to embodiments of the present invention may be administered by any appropriate route which results in an effective treatment in the living organism in need thereof.

In the preparation of pharmaceutical doses of the GS inhibitor composition for oral administration, the GS inhibitor composition may be mixed with solid, powdered ingredients, such as lactose, saccharose, sorbitol, mannitol, starch, amylopectin, cellulose derivatives, gelatin, and/or another suitable ingredient, as well as with disintegrating agents and lubricating agents such as magnesium stearate, calcium stearate, sodium stearyl fumarate and/or polyethylene glycol waxes. The mixture may then be processed into granules or pressed into tablets.

The following Examples are presented for illustrative purposes only, and do not limit the scope or content of the present application.

### EXAMPLE 1

To identify IMiD-modulated substrates of CRBN, a human embryonic kidney (HEK) 293T cell line was generated that stably expresses CRBN with a Flag tag appended to its amino terminus (^{Flag}CRBN). ^{Flag}CRBN cells were grown in medium formulated with isotopically light lysine and arginine (light medium) or in medium formulated with isotopically heavy lysine and arginine (heavy medium). Cells growing in heavy medium were treated with 50 μM thalidomide for 4 hours, whereas cells growing in light medium were treated with DMSO. ^{Flag}CRBN immunoprecipitates were prepared in parallel, mixed, and analyzed by quantitative mass spectrometry. Comparison of heavy:light ratios of peptides in the combined samples indicated that ^{Flag}CRBN, subunits of CRL4 (CUL4, DDB 1, RBX1), and subunits of the deneddylase enzyme CSN were recovered in equal amounts from cells treated with DMSO or thalidomide (FIG. 1). However, a number of putative substrates were recovered in lesser amounts from cells treated with thalidomide, as shown in Table 1, whereas one protein, glutamine synthetase (GS), was recovered in greater amounts (FIG. 1). Essentially identical results were obtained in a label swap experiment. The GS finding was of particular interest because recent work has shown that the level of CRBN expression correlates positively with IMiD sensitivity, suggesting that IMiDs might act through CRBN, as opposed to inhibiting CRBN. (Lopez-Girona et al., 2012, supra and Zhu et al. 2011 supra.)

TABLE 1

| | +/−Thalido- |
|---|---|
| Gene names | mide Ratio |
| RUVBL2 | 0.692702735 |
| KIF11 | 0.673725446 |
| WDR77 | 0.589527623 |
| GLNS | 2.508063449 |
| RUVBL1 | 0.709889429 |
| SNRPD3 | 0.658296855 |
| PIH1D1 | 0.515270413 |
| HNRNPK; HNRPK | 0.590544635 |
| BOLA2 | 0.598857168 |
| IVNS1ABP | 0.613076831 |
| TRIM21 | 0.712479811 |
| FUS | 0.580689474 |
| STK38 | 0.744495026 |
| COMT | 0.548219847 |
| CMBL | 0.746345707 |
| VCP; DKFZp434K0126 | 0.77019844 |
| SF3A3 | 0.595900095 |
| HSP90AA1 | 0.745441526 |
| PUF60 | 0.547071669 |
| SFRS11; SRSF11 | 0.572955238 |
| SNRPF | 0.60714932 |
| HNRNPU; HNRPU | 0.713744524 |
| CLNS1A | 0.626981496 |
| HSP90AB1 | 0.789986397 |
| SNRPD1 | 0.616791668 |
| C22orf28 | 0.710856769 |
| RPL26; KRBA2; RPL26L1 | 0.697997709 |
| U2AF2 | 0.612315329 |
| SPIN1 | 0.668859796 |
| TCP1 | 0.892651756 |
| PRPS2 | 0.644522087 |
| SNRPN; SNRPB | 0.66049843 |
| TXN | 0.703663751 |
| CCT8 | 0.836829057 |
| SRSF3; SFRS3 | 0.650957681 |
| DKFZp686K23100; MATR3; DKFZp686K0542 | 0.525988372 |
| PRPS1 | 0.726353702 |
| HNRNPL | 0.439962795 |
| RPL35; LOC154880 | 0.683910758 |
| DDX5; DKFZp686J01190 | 0.632144562 |
| HNRNPC; hCG_1641229; HNRPCL1; HNRNPCL1; | 0.422945565 |
| LOC440563; LOC649330 | |
| RPSA; RPSAP58; LAMR1P15 | 0.800547102 |
| HNRNPA2B1 | 0.453275527 |
| C12orf23 | 0.671822923 |
| ATP5B | 0.623583663 |
| IMPDH2 | 0.813783598 |
| C11orf84 | 0.703209501 |
| RPS25 | 0.73481289 |
| RIOK1 | 0.618678291 |
| GLUD1; GLUD2 | 0.793470294 |
| LUC7L2 | 0.757764973 |
| RBM39; DKFZp781C0423; DKFZp686A11192; | 0.618210651 |
| DKFZp781I1140; DKFZp686C17209 | |
| BAG2 | 0.6302998 |
| RPL27 | 0.798727238 |
| RBMX; RBMXL1 | 0.500368967 |
| CCT5 | 0.858384639 |
| PDIA6 | 0.851535402 |
| RPS8 | 0.818644799 |
| CCT6A | 0.848325712 |
| RPS3A | 0.76069131 |
| HNRNPAB | 0.569043974 |
| ATP5A1 | 0.754135965 |
| HNRNPM; ORF; HNRPM | 0.697658496 |

### EXAMPLE 2

To confirm the results of the quantitative mass spectrometry, ^{Flag}CRBN was immunoprecipitated from the stable HEK293T cell line and immunoblotted for CRL4^{CRBN} subunits and GS. As expected, thalidomide enhanced recovery of GS but had no effect on binding of DDB1 or CUL4A (FIG. 2). In order to confirm this finding, endogenous proteins expressed in cells were analyzed that are responsive to the therapeutic effects of IMiDs. For this purpose, endogenous CRBN as immunoprecipitated from MM.1S multiple myeloma cells. As seen in FIG. 3, the closely-related thalidomide derivative lenalidomide enhanced the recovery of endogenous GS in association with endogenous CRBN.

### EXAMPLE 3

The enhanced binding of GS to CRBN in cells treated with IMiDs suggested that IMiDs might modulate the ubiquitylation and/or subsequent degradation of GS. To evaluate ubiquitylation of endogenous GS, HEK293T cells were treated with DMSO, lenalidomide, or the proteasome inhibitor MG132, and then enriched ubiquitin conjugates on an ubiquitin-binding TUBE2 resin Immunoblotting of the bound fraction with antibodies against GS or ubiquitin revealed that lenalidomide caused an increase in GS ubiquitin conjugates similar to the increase that occurs upon blocking degradation of ubiquitin conjugates with the proteasome inhibitor MG132 (FIG. 4). However, lenalidomide had essentially no effect on the total cellular pool of ubiquitin conjugates.

### EXAMPLE 4

The consequences of IMiD-enhanced ubiquitylation of GS on endogenous GS levels and stability in MM.1S cells were evaluated. As shown in FIG. 5, both pomalidomide and lenalidomide caused a marked, dose-dependent reduction in the steady-state level of GS. The IMiD-triggered decline in GS protein was not due to a reduction in the mRNA level of GS (FIGS. 5 and 6). If IMiDs were added immediately after changing the growth medium, the suppression of GS was more profound, with a maximal effect seen at 0.1 μM pomalidomide (4-amino-thalidomide) (FIG. 7). Notably, the isomeric, clinically inactive 5-amino-thalidomide had no effect on GS protein levels (5-NH₂; FIG. 7). The suppression of GS caused by IMiDs was rapid, with a substantial reduction observed within 4 hours of adding lenalidomide (FIG. 8). To determine if IMiDs were affecting the stability of GS, cycloheximide chase experiments were performed. As known in the art, GS is an unstable protein (FIG. 9). However, the half-life of GS was decreased further by addition of either pomalidomide (FIG. 9) or lenalidomide (FIG. 10). The lenalidomide-induced downregulation of GS was blocked by the proteasome inhibitor MG132 (FIG. 11). Additionally, the lenalidomide-induced downregulation of GS was blunted upon depletion of CRBN by shRNA (FIG. 12).

### EXAMPLE 5

As mentioned above, GS is moderately unstable even in the absence of IMiDs. Specifically, seven lysine residues of GS are modified with ubiquitin in proteasome-inhibited cells, and modification of five of these sites is reduced upon inhibition of CRL activity with the NEDD8 conjugation inhibitor MLN4924. (Kim et al., 2011, Mol Cell., 44:325 and Emanuele et al., 2011, Cell, 147:459, the entire contents of both of which are herein incorporated by reference.) As such, the idea that the constitutive ubiquitylation and degradation of GS may be dependent on CRL4^{CRBN} was tested. In co-transfection assays, ^{HA}ubiquitin was incorporated into ^{Flag}GS as determined by immunoprecipitation of ^{Flag}GS followed by immunoblotting with anti-HA (FIG. 13A). Additionally, ubiquitin-modified ^{Flag}GS accumulated in cells in which the proteasome was inhibited with the proteasome inhibitor MG132, but was almost entirely absent upon depletion of endogenous CRBN (depletion of CRBN was confirmed by immunoblot; FIG. 13B). The CRBN-dependent ubiquitylation observed in FIG. 13A is likely to be direct, because binding was observed of the recombinant proteins produced in E. coli (FIG. 14), and endogenous GS co-immunoprecipitated from 293T cells with Flag-tagged CRL4^{CRBN} formed methylubiquitin-sensitive high molecular weight species (marked by asterisks in FIG. 15) when incubated in vitro with E1, E2, ubiquitin, and ATP. Finally, the steady-state level of GS was elevated in cells depleted of CRBN (FIG. 16). Additionally, there was detection of an effect of IMiDs on the direct association of recombinant GS and CRBN (FIG. 14) or on in vitro ubiquitylation reactions, suggesting that some covalent modification or cellular factor was required to mediate the effect of IMiDs on GS.

### EXAMPLE 6

To discern the impact of IMiD-induced degradation of GS on proliferation of myeloma cells, the consequences of GS knockdown were evaluated. Six different lentiviral shRNA constructs designed to deplete GS mRNA were generated and transduced into HEK293T and MM.1S cells, which were selected in puromycin for 5 days prior to analysis of GS levels by immunoblot (FIGS. 17A, 17B). Surprisingly, all GS-depleted MM.1S cultures were devoid of cells after 21 days even though they were grown in standard tissue culture medium that contained 2 mM glutamine. Thus, GS is essential for survival of MM.1S cells even when they are supplied with extracellular glutamine. When cell proliferation was monitored after transduction of GS shRNA-3 into MM.1S cells, the cell numbers ceased increasing 7 days after the puromycin selection (FIG. 18).

### EXAMPLE 7

Since the physiological concentration of glutamine in human plasma is 0.5 mM (not the 2 mM that is typically used in cell cultures), the impact of GS depletion on cells grown in 0.5 mM glutamine was evaluated. Cells transduced with GS shRNA-3 in 2 mM glutamine for 7 days and then the cells were shifted to 0.5 mM glutamine. After 48 hours, the GS-depleted cells displayed abnormal morphology with a granulated appearance characteristic of dying cells (FIG. 19). Quantification of cell death by staining with trypan blue revealed that more than 50% of MM.15 cells depleted of GS died within two days of the shift to 0.5 mM glutamine (FIG. 20) and exhibited elevated accumulation of cleaved caspase-3 and PARP (FIG. 21).

### EXAMPLE 8

Considering the knockdown experiments indicated that GS is essential for proliferation and viability of myeloma cells, it was reasoned that degradation of GS induced by IMiDs might account for their anti-myeloma activity. However, overexpression of GS from a lentivirus did not render cells resistant to lenalidomide. If IMiDs modulate the level of multiple proteins that are critical for proliferation of myeloma cells, then restoring any one protein would not be expected to allow proliferation in the presence of IMiDs. By contrast, it was reasoned that if degradation of GS contributes to the anti-myeloma effect of IMiDs, that reduction of GS levels by shRNA knockdown would enhance the effect of IMiDs at sub-saturating concentrations, resulting in enhanced efficacy. This is analogous to the relatively common observation that cells with low levels of a drug target are typically more sensitive to the drug because there is less target that needs to be inhibited, as has been reported for the response of proteasome-depleted cells to bortezomib (1). Indeed, cells depleted of GS using any one of three different shRNAs (shGS_{—}3, shGS_{—}4, and shGS_{—}5) and shifted to the physiological glutamine concentration (0.5 mM) showed significantly enhanced sensitivity to lenalidomide (FIGS. 22, 23) or pomalidomide (FIGS. 24, 25). These effects were observed at clinically-relevant doses of lenalidomide (0.5 μM; Cₘₐₓ is ˜1.5 μM) and pomalidomide (50 nM; Cₘₐₓ is ˜275 nM). (Blum et al., 2010, J. Clin Oncol, 28:4919, and Celgene, 2013, FDA package insert containing full prescribing information for Pomalyst, the entire contents of both of which are herein incorporated by reference.) A full dose-response emphasized the potent effect of GS depletion on accentuating the anti-myeloma activity of lenalidomide (FIG. 23) and pomalidomide (FIG. 25). The GS-depleted cells exhibited elevated PARP cleavage that was further accentuated at low concentrations of pomalidomide that had no effect on non-depleted cells (FIG. 26).

### EXAMPLE 9

Materials and cell lines. Thalidomide (Tocris Cookson), lenalidomide (Chem-Pacific) and pomalidomide (Selleck Chemicals) were dissolved in dimethylsulfoxide (DMSO) at room temperature to make a 50 mM stock solution and were stored at −80° C. until use.

MM.1S, a human multiple myeloma (MM) cell line was purchased from ATCC (American Type Culture Collection, Manassas, Va., USA). MM.1S Cells were maintained in RPMI-1640 medium containing 10% (v/v) heat-inactivated fetal bovine serum (Gibco, Grand Island, N.Y., USA) supplemented with 2 mM glutamine and penicillin-streptomycin. HEK-293T cells were purchased from ATCC and were grown in DMEM supplemented with 10% FBS and penicillin-streptomycin.

### EXAMPLE 10

Plasmids. Human CRBN and GS expression vectors pCMV6-CRBN-Myc-Flag and pCMV6-GS-Myc-Flag (C-terminal Myc- and Flag-tagged) were purchased from OriGene. pCMV6-GS-Myc was generated by introducing a STOP codon between Myc and Flag by using a QuikChange Site-Directed Mutagenesis Kit (Stratagene, La Jolla, Calif.). pcDNA3-HA2-RBX1 was kindly provided by Dr. Yue Xiong (University of North Carolina at Chapel Hill). pcDNA3-HA2-DDB1 was from Addgene (19909). pcDNA3-CUL4A-V5 as described in Besten et al.,2012, Nature Structural & Molecular Biology, 19:511, the entire contents of which are herein incorporated by reference.

### EXAMPLE 11

Generation of stable HEK293T cells expressing ^{Flag}CRBN. To make a lentiviral vector directing expression of wild-type ^{Flag}CRBN, CRBN was constructed in pCDH-T2AcGFP-MSCV (System Biosciences). The lentiviruses were precipitated using PEG-it Virus Precipitation Solution according to the manufacturer's protocol (System Biosciences). Infection efficiency was >95% as judged by fluorescence microscopy and CRBN expression was confirmed by Western blot.

### EXAMPLE 12

Preparation of ^{Flag}CRBN for mass spectrometry (MS). SILAC-labeled cultures of the stable cell lines were grown as described Lee et al., 2011, Molecular & Cellular Proteomics, 10: M110 006460,the entire contents of which are herein incorporated by reference. Briefly, HEK293T cells, stably expressing ^{Flag}CRBN, were cultured in medium formulated with isotopically light lysine and arginine (‘light’ medium) or in medium formulated with isotopically heavy lysine and arginine (‘heavy’ medium). The cells growing in ‘heavy’ medium were treated with thalidomide (a final concentration of 50 μM) for 4 hours, while the cells growing in ‘light’ medium were treated with DMSO. SILAC experiments were repeated after swapping the SILAC labels in which the cells cultured in ‘light’ and ‘heavy’ medium were treated with thalidomide and DMSO, respectively. The ^{Flag}CRBN immunoprecipitates were prepared, mixed, and analyzed by quantitative mass spectrometry.

### EXAMPLE 13

MS data analysis. Thermo raw files were processed and searched with MaxQuant (v. 1.4.1.2), as described in Cox et al., 2008, Nat. Biotechnol. 26:1367; and Cox et al., 2011, J. of Proteome Research, 10:1794, the entire contents of both of which are herein incorporated by reference. Trypsin was specified as the digestion enzyme with up to two missed cleavages. Protein N-terminal acetylation (+42.0106) and methionine oxidation (+15.9949) were specified as variable modifications. Carbamidomethylation of cysteine (+57.0215) was specified as a fixed modification. Arg6 (+6.0138) and Lys8 (+8.0142) were specified as the SILAC labels. Requantification and match between runs were enabled. Precursor ion tolerance was 7 ppm and fragment ion tolerance was 0.5 Da. All human Uniprot entries were searched (148298 sequences, downloaded on 05Dec12) along with a contaminant database containing proteins such as keratin and trypsin (247 sequences). (Apweiler et al., 2013, Nucleic Acids Research, 41: D43, the entire contents of which are herein incorporated by reference.) Additionally, to determine the false discovery rate, a decoy database was constructed by reversing the target database. While no minimum score was specified, the protein and peptide level false discovery rates were fixed at 1% and we required that all proteins reported were identified in both biological replicates by at least two peptides.

Proteins were quantified by first calculating the median of all peptide ratios within each biological replicate and then calculating the mean of the two biological replicate ratios. Only peptides uniquely assignable to the protein group were used for quantification. Ratios were normalized in each biological replicate so that the bait (CRBN) had a ratio of 1. Individual ratio measurement error was estimated using pooled variance and overall ratio standard error was calculated using bootstrap analysis. P-values were calculated using a z-test where the null hypothesis was the protein was unchanged (i.e., had a ratio of 1). Q-values were calculated from the p-values using the Storey method for calculating false discovery rates as described in Storey et al., 2002, J. Roy Stat. Soc. B., 64:479, the entire contents of which is herein incorporated by reference. A q-value of 0.05 was used as a threshold for significance.

### EXAMPLE 14

Lentiviral shRNAs. The lentiviral constructs expressing nontargeting (control, CT), human CRBN shRNAs (CRBN_{—}1 shRNA: V2LHS_{—}226831; CRBN_{—}2 shRNA: V2LHS_{—}115329; CRBN_{—}3; shRNA: V2LHS_{—}224589; CRBN_{—}4 shRNA: V3LHS_{—}413798; CRBN_{—}5 shRNA: V3LHS_{—}395310), and human GLUL shRNAs (GLUL_{—}1 shRNA: V3LHS_{—}338700; GLUL_{—}2 shRNA: V3LHS_{—}338702; GLUL_{—}3 shRNA: V2LHS_{—}114133; GLUL_{—}4 shRNA: V2LHS_{—}114134; GLUL_{—}5 shRNA: V2LHS_{—}224778; GLUL_{—}6 shRNA: V3LHS_{—}338704) in the pGIPZ lentiviral vector were purchased from Open Biosystems. Five lentiviruses targeting CRBN or six lentiviruses targeting GLUL were screened to identify shRNAs that optimally suppressed CRBN or GS. Virus preparation and cell infection were performed according to the manufacturer's protocol, with minor modifications. Briefly, shRNA-encoding plasmids were co-transfected with psPAX2 (packaging plasmid) and pMD.2G (enveloping plasmid) into HEK293T cells using Fugene 6 (Roche). Virus-containing supernatants were harvested at 48 h and 72 h post transfection. The lentiviruses were precipitated using PEG-it virus precipitation solution according to the manufacturer's protocol (System Biosciences), and target cells were infected in the presence of 8 μg/ml polybrene. After 24 hours of transduction, the cells were selected with 1 μg/ml puromycin for 1 week, and then maintained in complete medium supplemented with 0.5 μg/ml puromycin. Knockdown efficiencies were analyzed by immunoblot at 5 days after transduction.

### EXAMPLE 15

Cell Viability Assays. MM.1S cells were cultured in 96-well plates with 2×10⁴ cells per well and treated with serial doses of lenalidomide or pomalidomide for 3 days. Cell viability was assessed using the Cell-Titer Glo kit (G7572; Promega) according to the protocol recommended by the manufacturer. All experiments were performed in triplicate and repeated at least twice.

Cell death was analyzed by measuring the permeability of the plasma membrane to Trypan blue. Non-target (control) or GS shRNA-expressing MM.1S cells were maintained in complete medium containing 2 mM glutamine. The cells were then shifted to medium containing 0.5 mM glutamine for 48 or 96 hours. Cells were washed in PBS and stained with 0.04% Trypan blue. The percentages of dead cells were evaluated by counting the number of Trypan Blue⁺ cells [% dead cells=(Trypan blue+positive cells/total cell number)*100].

### EXAMPLE 16

Antibodies. Anti-Flag (M2, F3165) was from Sigma. Anti-HA (influenza hemagglutinin) (16B12, MMS-101P) was from Covance. Anti-Myc (9E10) was from Santa Cruz Biotechnology. Anti-DDB1 (ab21080) was from Abcam. Anti-GST was from GE Healthcare Life Sciences. Anti-glutamine synthetase (C-20) (sc-6640-R) was from Santa Cruz Biotechnology. Antibodies to PARP (9542S), Cleaved Caspase-3 (Asp175; 9664S) and CUL4A (2699) were from Cell Signaling Technology. Mouse monoclonal anti-CRBN antibody against amino acids 1-18 of human CRBN was previously disclosed in Lopez-Girona et al.,2012, supra and Zhu et al., 2011, supra. Anti-GAPDH (MAB374) was from Millipore.

### EXAMPLE 17

Protein expression and purification of recombinant human CRBN. Human CRBN gene was cloned into pGEX-4T1 with an N-terminal GST tag followed by a TEV cleavage site and a Flag tag. Recombinant GST-^{Flag}CRBN was expressed in bacteria and purified by standard methods.

### EXAMPLE 18

GST pull-down. Recombinant ^{Flag}CRBN (2 μg) was mixed with 1 μg of GST protein (control) or GST-glutamine synthetase protein (H00002752-P01, Novus biologicals) in 500 μA of binding buffer (10 mM HEPES-KOH at pH 7.5, 100 mM potassium acetate at pH 7.5, 5 mM magnesium acetate, 0.5% NP-40, 1 mM dithiothreitol) for 1 h at 4° C., followed by incubation with 20 μl of glutathione-sepharose 4B (GE Healthcare) for 1 hr at 4° C. Beads were collected by centrifugation at 2,000 g for 1 min, washed three times with binding buffer and boiled in 2× SDS loading buffer. Samples were separated by 10% SDS-PAGE and transferred to PVDF membrane (Immobilon-P, Millipore). Proteins were detected using antibodies against GST and Flag.

### EXAMPLE 19

Western blot analysis. MM.1S cells were harvested after treatment with serial doses of lenalidomide or pomalidomide. After washing twice with ice-cold PBS, the cells were lysed in RIPA buffer (50 mM Tris-HCl, 150 mM NaCl, 1% Triton X-100, 1% Sodium deoxycholate, 0.1% SDS, [pH 7.5]) supplemented with complete protease inhibitor (Roche). Whole-cell protein extracts were prepared and quantified by the Bradford method (Bio-Rad, Hercules, Calif.). Equal amounts of protein (20-80 μg/lane) were electrophoretically separated on SDS-PAGE and transferred to a PVDF membrane. The membranes were blotted with indicated antibodies. Anti-rabbit or anti-mouse antibodies conjugated to horseradish peroxidase (Vector Labs) were used as secondary antibodies, and the signal was detected using a Super Signal West Pico Substrate kit (Fisher Scientific).

### EXAMPLE 20

Immunoprecipitation. Cells were lysed in immunoprecipitation buffer (10 mM Tris [pH 7.5], 150 mM NaCl, 1% Triton X-100) containing a protease inhibitor cocktail, and immunoprecipitated with the indicated antibodies, such as anti-CRBN antibody for 2-4 hours at 4° C., followed by incubation with protein G sepharose 4 Fast Flow (GE Healthcare) for 1 hour at 4° C. Immunoprecipitated proteins were resolved by SDS/PAGE and analyzed by immunoblotting with the indicated antibodies.

### EXAMPLE 21

Cycloheximide Chase Experiments. MM.1S cells were seeded overnight in complete medium in 24-well plates (1×10⁵ cells/well), and then pre-treated with pomalidomide or lenalidomide for 1 hour, followed by addition of 100 μg/ml cycloheximide (CHX). At the indicated times following addition of CHX, samples were harvested for immunoblot analysis.

### EXAMPLE 22

TUBE (Tandem Ubiquitin Binding Entities) Pull-down. HEK293T cells were seeded in a 6-well plate, and then treated with DMSO, lenalidomide (30 μM), or the proteasome inhibitor MG132 (10 μM) for 3 hours. The cells were lysed in immunoprecipitation buffer (10 mM Tris [pH 7.5], 150 mM NaCl, 1% Triton X-100) containing a protease inhibitor cocktail, MG132 and 10 mM N-ethylmaleimide (NEM, Sigma). Whole-cell protein extracts were incubated with 20 μl of TUBE2 agarose beads (Boston Biochem) for 2-4 hours with rotation at 4° C. Beads were washed 3-5 times with lysis buffer, and bound proteins were eluted in SDS sample buffer and subjected to Western blot analysis.

### EXAMPLE 23

In vitro Ubiquitylation assay. The assays were performed as described in Duan et al., 2012, Nature, 481:90 and Kleiger et al., 2009, Cell, 139:957, the entire contents of both of which are herein incorporated by reference. Briefly, HEK293T cells stably expressing ^{Flag}CRBN were transiently transfected with plasmids that expressed CUL4A^{V5}, ^{HA}DDBI and ^{HA}RBX1. For control samples, HEK293T cells transduced with lentiviral empty vector were transiently transfected with an empty vector. After 30 hrs of transfection, the cells were treated with MG 132 (10 μM) for 3 hours. Then, the cells were lysed in immunoprecipitation buffer and immunoprecipitated with anti-Flag M2 agarose beads for 2-4 hours at 4° C. After washing five times with IP lysis buffer and two times with ubiquitylation buffer (30 mM Tris-HCl [pH 7.6], 5 mM MgCl2, 100 mM NaCl, 1 mM DTT), the beads were incubated at 30° C. for 2 hours in 30 μl of ubiquitylation buffer containing E1 (0.5 μM), UbcH5a (0.5 μM), UbcH3 (1.67 μM), ubiquitin (60 μM), and 4 mM ATP. Reactions were stopped by adding SDS sample buffer and subjected to Western blot analysis.

### EXAMPLE 24

In vivo ubiquitylation assay. HEK293T stable cell lines expressing nontarget shRNA (Control, CT) or CRBN shRNAs (a combination of CRBN_{—}1 plus CRBN_{—}5) were transiently transfected with plasmids that expressed ^{Flag}GS (6 μg) and ^{HA}ubiquitin (3 μg) in 10-cm plates. After 30 hrs of transfection, the cells were treated with DMSO or MG 132 (10 μM) for 3 hours. Then, the cells were lysed in 0.3 ml denaturing IP lysis buffer (1% SDS, 50 mM Tris, 10 mM DTT, [pH 7.5]) and boiled for 5 minutes. Subsequently, denatured proteins were diluted 10× in immunoprecipitation buffer and immunoprecipitated with anti-Flag resin. IP washing steps were performed using IP lysis buffer supplemented with 0.5 M NaCl Immunoprecipitated proteins were resolved by SDS/PAGE and analyzed by immunoblotting.

### EXAMPLE 25

RNA Extraction and real time PCR assay. Total RNA was extracted using the RNeasy Mini Kit from QIAGEN and converted into cDNA using Advantage RT-for-PCR Kit (Clontech) according the protocols described in the handbooks. Quantitative RT-PCR was performed using TagMan gene expression assay (Applied Biosystems) and analyzed on the GeneAmp 7700 sequence detection system (Applied Biosystems). Gene expression was normalized to GAPDH mRNA level. The relative abundance is shown as an average of triplicates of quantitative PCR in each sample, and error bars indicate±SD. All primers were purchased from Applied Biosystems: human GAPD (GAPDH) Endogenous Control (4326317E), human glutamine synthetase (Hs00365928_gl).

As disclosed throughout and evidenced by the data presented in the accompanying figures, for example, FIGS. 22-25, the compositions of the present invention provide a means for inhibiting the proliferation of neoplastic growth such as cancer.

While the present invention has been illustrated and described with reference to certain exemplary embodiments, those of ordinary skill in the art will understand that various modifications and changes may be made to the described embodiments without departing from the spirit and scope of the present invention, as defined in the following claims.

What is claimed is:

1. A method of treating neoplastic growth, comprising:
administering a composition comprising a glutamine synthetase (GS) inhibitor to a subject having the neoplastic growth.

2. The method of claim 1, wherein the neoplastic growth comprises a cancer.

3. The method of claim 2, wherein the cancer comprises multiple myeloma, myeloma, bladder cancer, breast cancer, colon cancer, rectal cancer, endometrial cancer, renal cell cancer, leukemia, lung cancer, melanoma, non-Hodgkin's lymphoma, Hodgkin's lymphoma, pancreatic cancer, prostate cancer, or thyroid cancer.

4. The method of claim 2, wherein the cancer is myeloma.

5. The method of claim 1, further comprising administering thalidomide, lenalidomide and/or pomalidomide.

6. The method of claim 1, wherein the glutamine synthetase inhibitor comprises GS anti-sense mRNA, GS siRNA, GS shRNA, GS miRNA, and/or GS oligonucleotides.

7. The method of claim 6, wherein the glutamine synthetase inhibitor comprises GS shRNA.

8. The method of claim 1, wherein the glutamine synthetase inhibitor comprises methionine sulfoximine, methionine sulfone, phosphinothricin, tabtoxinin-b-lactam, methionine sulfoximine phosphate, alpha-methyl methionine sulfoximine, alpha-ethyl methionine sulfoximine, ethionine suloximine, alpha-methyl ethionine sulfoximine, prothionine sulfoximine, alpha-methyl prothionine sulfoximine, gamma-hydroxy phosphinothricin, gamma-methyl phosphinothricin, gamma-acetoxy phosphinothricin, alpha-methyl phosphinothricin, alpha-ethyl phosphinothricin, cyclohexane phosphinothricin, cyclopentane phosphinothricin, tetrhydrofuran phosphinothricin, s-phosphonomethylhomocysteine, s-phosphonomethyl homocysteine sulfoxide, s-phosphonomethyl homocysteine sulfone, 4-(phosphonoacetyl)-L-alpha-aminobutyrate, threo-4-hydroxy-D-glutamic acid, threo-4-fluoro-D,L-glutamic acid, erythro-4-fluoro-D,L-glutamic acid, 2-amino-4-[(phosphonomethyl) hydroxyphosphinyl)]butanoic acid, alanosine, 2-amino-4-phosphono butanoic acid, 2-amino-2-methyl-4-phosphono butanoic acid, 4-amino-4-phosphono butanoic acid, 4-amino-4-(hydroxymethylphosphinyl) butanoic acid, 4-amino-4-methyl-4-phosphono butanoic acid, 4-amino-4-(hydroxymethylphosphinyl)-4-methyl butanoic acid, 4-amino-4 phosphono butanamide, 2-amido-4-phosphono butanoic acid, 2-methoxycarbonyl-4-phosphono butanoic acid, methyl 4-amino-4-phosphono butanoate, oxetin, IF7 peptide, and/or IF17 peptide.

9. A method of identifying a response to immunomodulatory drug (IMiD) and glutamine synthetase (GS) inhibitor therapy in a subject, comprising:
measuring the level of glutamine synthetase (GS) protein in the subject before administering the IMiD and GS inhibitor therapy to the subject;
administering IMiD and GS inhibitor therapy to the subject; and
measuring the level of GS protein in the subject after administering the IMiD and GS inhibitor therapy, wherein a reduction in the level of GS protein in the subject after IMiD and GS inhibitor therapy is indicative of a response to the IMiD therapy.

10. The method of claim 9, wherein the immunomodulatory drug therapy comprises thalidomide, lenalidomide and/or pomalidomide.

11. A method of identifying the capability of a subject having neoplastic cell growth to respond to immunomodulatory drug therapy, the method comprising:
determining the amount of glutamine synthetase expression in the neoplastic cell growth of the subject;
determining the amount of glutamine synthetase expression in normal cells of the subject; wherein an increase in the expression of glutamine synthetase in the neoplastic cell growth compared to the expression of glutamine synthetase in normal cells of the subject indicates that the neoplastic cell growth of the subject is capable of responding to immunomodulatory drug therapy.

12. A composition for inhibiting neoplastic cell growth, comprising:
thalidomide, lenalidomide and/or pomalidomide; and
a glutamine synthetase inhibitor.

13. The composition of claim 12, wherein the glutamine synthetase inhibitor comprises GS anti-sense mRNA, GS siRNA, GS shRNA, GS miRNA, and/or GS oligonucleotides.

14. The composition of claim 12, wherein the glutamine synthetase inhibitor comprises the group consisting of methionine sulfoximine, methionine sulfone, phosphinothricin, tabtoxinin-b-lactam, methionine sulfoximine phosphate, alpha-methyl methionine sulfoximine, alpha-ethyl methionine sulfoximine, ethionine suloximine, alpha-methyl ethionine sulfoximine, prothionine sulfoximine, alpha-methyl prothionine sulfoximine, gamma-hydroxy phosphinothricin, gamma-methyl phosphinothricin, gamma-acetoxy phosphinothricin, alpha-methyl phosphinothricin, alpha-ethyl phosphinothricin, cyclohexane phosphinothricin, cyclopentane phosphinothricin, tetrhydrofuran phosphinothricin, s-phosphonomethylhomocysteine, s-phosphonomethyl homocysteine sulfoxide, s-phosphonomethyl homocysteine sulfone, 4-(phosphonoacetyl)-L-alpha-aminobutyrate, threo-4-hydroxy-D-glutamic acid, threo-4-fluoro-D,L-glutamic acid, erythro-4-fluoro-D,L-glutamic acid, 2-amino-4-[(phosphonomethyl)hydroxyphosphinyl)]butanoic acid, alanosine, 2-amino-4-phosphono butanoic acid, 2-amino-2-methyl-4-phosphono butanoic acid, 4-amino-4-phosphono butanoic acid, 4-amino-4-(hydroxymethylphosphinyl) butanoic acid, 4-amino-4-methyl-4-phosphono butanoic acid, 4-amino-4-(hydroxymethylphosphinyl)-4-methyl butanoic acid, 4-amino-4 phosphono butanamide, 2-amido-4-phosphono butanoic acid, 2-methoxycarbonyl-4-phosphono butanoic acid, methyl 4-amino-4-phosphono butanoate, oxetin, IF7 peptide, and/or IF17 peptide.

15. The composition of claim 12, wherein the neoplastic growth comprises a cancer.

16. The composition of claim 12, wherein the cancer comprises multiple myeloma, myeloma, bladder cancer, breast cancer, colon cancer, rectal cancer, endometrial cancer, renal cell cancer, leukemia, lung cancer, melanoma, non-Hodgkin lymphoma, Hodgkin's lymphoma, pancreatic cancer, prostate cancer, or thyroid cancer.

17. The composition of claim 15, wherein the cancer comprises myeloma.
